(19) United States
(12) Patent Application Publication
(10) Pub. No.: US 2025/0288700 A1
(43) Pub. Date: Sep. 18, 2025

(54) METHOD FOR EVALUATING A COSMETIC COMPOSITION OR COMPONENT THEREOF

(71) Applicant: Conopco, Inc., d/b/a UNILEVER, Englewood Cliffs, NJ (US)

(72) Inventors: Luonan CHEN, Shanghai (CN); Chung-Ching CHU, Shanghai (CN); Mingming PU, Shanghai (CN); Lu WANG, Tianjin (CN); Yining XU, Shanghai (CN)

(73) Assignee: Conopco, Inc., d/b/a UNILEVER, Englewood Cliffs, NJ (US)

(21) Appl. No.: 18/575,997

(22) PCT Filed: Jul. 1, 2022
(86) PCT No.: PCT/EP2022/068286

§ 371 Date: Jan. 2, 2024

(30) Foreign Application Priority Data
Jul. 5, 2021 (WO) PCT/CN2021/104606
Sep. 2, 2021 (EP) 21194474.9

(51) Int. Cl. A61K 49/00; A61K 8/99; A61Q 19/00; C12Q 1/689

(52) U.S. Cl. CPC A61K 49/0006; A61K 8/99; A61Q 19/00; C12Q 1/689; C12Q 2600/148; C12Q 2600/156

(57) ABSTRACT

Disclosed is a method for evaluating a cosmetic composition or a component thereof, comprising the steps of: (i) identifying a first individual microbiome network on a skin surface of a person; (ii) treating the skin surface having the identified first individual microbiome network with a cosmetic composition or the component thereof; (iii) identifying a second individual microbiome network on the skin surface that was treated; and (iv) comparing the first and second individual microbiome networks.

### FIELD OF THE INVENTION

The present invention relates a method for evaluating a cosmetic composition or component thereof. In particular, such method is especially sensitive for demonstrating the efficiency of the cosmetic composition or component thereof.

### BACKGROUND OF THE INVENTION

Cosmetic compositions which improve the condition of the skin or hair are often designed to do so by influencing the ability of the skin or hair by providing different benefits to skin or hair. Unfortunately, however, the benefits of such products may not be perceived by a user, especially those having heathy conditions.

Our skin harbours a diverse range of microorganisms, which live as a community and function as a component of skin biology. It is believed that the microbiome plays a partial role in maintaining a healthy skin function, while the diversity, composition, and stability of skin microbiomes may also be influenced by host intrinsic factors for example genetics, or environmental factors. Network theory has been employed as a promising approach to analyze the complex skin microbiome with multifaceted interactions between microbiotas.

The present inventors have recognized that there is a need for developing a sensitive evaluation method to evaluate the efficiency of a cosmetic composition. Therefore, they have developed a method for evaluating a cosmetic composition or a component thereof, utilizing the individual microbiome network of a skin surface of person. It was surprisingly found that the method is very sensitive and capable of demonstrating the efficiency of the cosmetic composition or a component thereof.

### SUMMARY OF THE INVENTION

In a first aspect, the present invention is directed to a method for evaluating a cosmetic composition or a component thereof, comprising the steps of: (i) identifying a first individual microbiome network on a skin surface of a person; (ii) treating the skin surface having the identified first individual microbiome network with a cosmetic composition or the component thereof; (iii) identifying a second individual microbiome network on the skin surface that was treated; and (iv) comparing the first and second individual microbiome networks, wherein identifying a first individual microbiome network comprises the steps of: (a) obtaining a first population microbiome network from a group of samples consisting of N individuals including said person, wherein N is an integer at least 3; (b) obtaining a second populational microbiome network from the same group of samples without the sample from said person; and (c) determining the first individual microbiome network of said person by subtracting the effects of the second population microbiome network from the first population microbiome network.

In a second aspect, the present invention is directed to a method for preparing a recordable medium for demonstrating the evaluating result of a cosmetic composition or a component thereof, the method comprising the steps of: (i) identifying a first individual microbiome network on a skin surface of a person; (ii) treating the skin surface having the identified first individual microbiome network with a cosmetic composition or the component thereof; (iii) identifying a second individual microbiome network on the skin surface that was treated; (iv) comparing the first and second individual microbiome networks; and (v) capturing one or more images for the comparison and storing the same on a recordable medium, wherein identifying a first individual microbiome network comprises the steps of: (a) obtaining a first population microbiome network from a group of samples consisting of N individuals including said person, wherein N is an integer at least 3; (b) obtaining a second populational microbiome network from the same group of samples without the sample from said person; and (c) determining the first individual microbiome network of said person by subtracting the effects of the second population microbiome network from the first population microbiome network.

In a third aspect, the present invention is directed to a method for demonstrating the evaluating result of a cosmetic composition or a component thereof comprising a step of displaying images of microbiome network, wherein the images are captured and stored for the following process; (i) identifying a first individual microbiome network on a skin surface of a person; (ii) treating the skin surface having the identified first individual microbiome network with a cosmetic composition or the component thereof; (iii) identifying a second individual microbiome network on the skin surface that was treated; and (iv) comparing the first and second individual microbiome networks, wherein identifying a first individual microbiome network comprises the steps of: (a) obtaining a first population microbiome network from a group of samples consisting of N individuals including said person, wherein N is an integer at least 3; (b) obtaining a second populational microbiome network from the same group of samples without the sample from said person; and (c) determining the first individual microbiome network of said person by subtracting the effects of the second population microbiome network from the first population microbiome network.

All other aspects of the present invention will more readily become apparent upon considering the detailed description and examples which follow.

### DETAILED DESCRIPTION OF THE INVENTION

Except in the examples, or where otherwise explicitly indicated, all numbers in this description indicating amounts of material or conditions of reaction, physical properties of materials and/or use may optionally be understood as modified by the word “about”.

All amounts are by weight of the composition, unless otherwise specified.

It should be noted that in specifying any range of values, any particular upper value can be associated with any particular lower value.

For the avoidance of doubt, the word “comprising” is intended to mean “including” but not necessarily “consisting of” or “composed of”. In other words, the listed steps or options need not be exhaustive.

The disclosure of the invention as found herein is to be considered to cover all embodiments as found in the claims as being multiply dependent upon each other irrespective of the fact that claims may be found without multiple dependency or redundancy.

Where a feature is disclosed with respect to a particular aspect of the invention (for example a composition of the invention), such disclosure is also to be considered to apply to any other aspect of the invention (for example a method of the invention) mutatis mutandis.

“Microbiome” as used herein refers to the diverse ecological community of commensal bacteria, fungi, viruses, and/or parasites that are associated with an organism.

“Microbiome network” as used herein refers to a co-occurrence network built through network theory of microbiome abundance data, indicating the direct interactions and/or indirect interactions among the microbiomes.

“Population Microbiome network” as used herein refers to the microbiome network of a group of samples, which typically reflects the interactions among the group of samples. Population microbiome network is exchangeable with microbiome network of population which may be abbreviated as MNP.

“Individual microbiome network” as used herein refers to the microbiome network of a single sample. Individual microbiome network is exchangeable with microbiome network of individual which may be abbreviated as MNI.

To fully understand the preferred embodiment of the present invention, the network analysis of microbiome will be described. Network models can portray the members of a microbial community along with inference about their interactions. The microbiome network is usually visualized by a set of nodes connected to each other by many edges. “Nodes” as used herein refer to individual entities that are the building blocks of a microbiome network, typically represents a microbiome taxonomy feature such as an amplicon sequence variant (ASV), operational taxonomic unit (OTU), a microbial species, or a microbial genus. “Edges”, as used herein refers to the connections between nodes in a network, which reflect the association, relation, and interaction between the nodes. “Node degree” as used herein refers to the number of edges between itself and other nodes. The node degree is usually used to describe the connectivity of the network. The frequency distribution of node degree is usually used to infer network robustness. “Clustering coefficient” as used herein refers to ratio of the number of edges between the neighbors of a node, and the maximum number of edges that could possibly exist between its neighbors. The clustering coefficient of a node is always a number between 0 and 1.

The way of identifying the first individual microbiome network of a person comprises the steps of: (a) obtaining a first population microbiome network from a group of samples consisting of N individuals including said person, wherein N is an integer at least 3; (b) obtaining a second populational microbiome network from the same group of samples without the sample from said person, i.e. N−1 samples; and (c) determining the first individual microbiome network of said person by subtracting the effects of the second population microbiome network from the first population microbiome network. Preferably, N is an integer of at least 10, more preferably at least 20, even more preferably 35 to 1,000,000, and most preferably 50 to 100,000.

Preferably, the method of obtaining a population microbiome network comprises the step of determining microbiome composition data of the skin surface; and generating a population microbiome network by representing microbial organisms in each matrix as a network of plurality of nodes corresponding the group of samples.

Preferably, the way of determining microbiome composition data comprises collecting microbial samples from the skin surfaces of a group of persons, preferably by tape stripping, swabbing, or buffer scrubbing, or any other methods suitable for body surface microbe collection; extracting DNA using any established methods for each sample; and sequencing of DNA samples by a sequencer to generate a plurality of DNA sequences. Preferably, the way of determining microbiome composition data further comprises the step of creating a matrix of microbial abundance profile of the operational taxonomic unit (OTU), amplicon sequence variant (ASV) or further accumulating at different taxonomy level corresponding to each person.

The population microbiome network may be generated in any suitable way. However, it is preferable that population microbiome networks are constructed by MENA (Molecular Ecological Network Analysis), LSA (local similarity analysis), SparCC (Sparse Correlations for Compositional data) and NetCoMi (Network Construction and comparison for Microbiome data). It is more preferable that population microbiome networks are constructed by SParse InversE Covariance estimation for Ecological Association Inference (SPIEC-EASI) analysis from the matrix of sequencing counts. Preferably, the method of obtaining a population microbiome network further comprises the step of visualizing the relationships of a set of nodes with edges.

Preferably, the microbiome composition of the present invention comprises a set of taxa comprising at least one of: Marvinbryantia (genus), Eysipelotrichales (order), Erysipelotrichia (class), Bacteroidetes (phylum), Staphylococcus (genus), Staphylococcaceae (family), Bacillales (order), Actinobacteria (class), Firmicutes (phylum), Actinobacteria (phylum), and Cutibacterium.

“Skin” as used herein is meant to include skin on the face, oral and body (e.g., neck, chest, back, arms, underarms, hands, legs, buttocks and scalp). The skin surface is selected from any surface of body skin and/or face skin. Preferably, the skin surface is selected from scalp or face skin. Most preferably, the skin surface is selected from face skin, in particular cheek. It should be noted that the sample is from the same body site of N individuals including said person.

Preferably, the skin surface has an undesired skin condition, preferably the undesired skin condition is selected from dry skin, aging, wrinkle, darkness, acne, spots, dandruff and/or weak skin barrier.

“Cosmetic composition” refers to any product applied to a human body for improving appearance, sun protection, reducing wrinkled appearance or other signs of photoaging, odor control, skin lightening, even skin tone, or general aesthetics. Non-limiting examples of cosmetic compositions include lotions, creams, facial masks, gels, sticks, antiperspirants, deodorants, liquid or gel body washes, soap bars, oral care products, and sunless tanners.

The composition preferably comprises a surfactant. More than one surfactant may be included in the composition. The surfactant may be chosen from soap, non-soap anionic, cationic, non-ionic, amphoteric surfactant and mixtures thereof. Many suitable surface-active compounds are available and are fully described in the literature, for example, in “Surface-Active Agents and Detergents”, Volumes I and II, by Schwartz, Perry and Berch. The preferred surfactant that can be used are soaps, non-soap anionic, non-ionic surfactant, amphoteric surfactant or a mixture thereof.

Suitable non-soap anionic surfactants include linear alkylbenzene sulphonate, primary and secondary alkyl sulphates, particularly C₈ to C₁₅ primary alkyl sulphates; alkyl ether sulphates; olefin sulphonates; alkyl xylene sulphonates; dialkyl sulphosuccinates; fatty acid ester sulphonates; or a mixture thereof. Sodium salts are generally preferred.

Most preferred non-soap anionic surfactant are linear alkylbenzene sulphonate, particularly linear alkylbenzene sulphonates having an alkyl chain length of from C₈ to C₁₅. It is preferred if the level of linear alkylbenzene sulphonate is from 0 wt % to 30 wt %, more preferably from 1 wt % to 25 wt %, most preferably from 2 wt % to 15 wt %, by weight of the total composition.

Nonionic surfactants that may be used include the primary and secondary alcohol ethoxylates, especially the C₈ to C₂₀ aliphatic alcohols ethoxylated with an average of from 1 to 20 moles of ethylene oxide per mole of alcohol, and more especially the C₁₀ to C₁₅ primary and secondary aliphatic alcohols ethoxylated with an average of from 1 to 10 moles of ethylene oxide per mole of alcohol. Non ethoxylated nonionic surfactants include alkylpolyglycosides, glycerol monoethers, and polyhydroxyamides (glucamide). It is preferred if the level of non-ionic surfactant is from 0 wt % to 30 wt %, preferably from 1 wt % to 25 wt %, most preferably from 2 wt % to 15 wt %, by weight of a fully formulated composition comprising the microcapsules of the invention.

Suitable amphoteric surfactants preferably are betaine surfactants. Examples of suitable amphoteric surfactants include, but are not limited to, alkyl betaines, alkylamido betaines, alkyl sulfobetaines, alkyl sultaines and alkylamido sultaines; preferably, those having 8 to about 18 carbons in the alkyl and acyl group. It is preferred that the amount of the amphoteric surfactant is 0 to 20 wt %, more preferably from 1 to 10 wt %, by weight of the composition.

Water-insoluble skin benefit agents may also be formulated into the compositions as conditioners and moisturizers. Examples include silicone oils; hydrocarbons such as liquid paraffins, petrolatum, microcrystalline wax, and mineral oil; and vegetable triglycerides such as sunflower seed and cottonseed oils.

The composition may comprise optional ingredients including pigment, moisturizing agent, organic sunscreen, skin glowing agent, fragrance, natural extract, or a combination thereof.

Pigments suitable for the present inventions are typically particles of refractive index materials greater than 1.3, more preferably greater than 1.8 and most preferably from 2.0 to 2.7. Examples of such pigment are those comprising bismuth oxy-chloride, boron nitride, barium sulfate, mica, silica, titanium dioxide, zirconium oxide, aluminium oxide, zinc oxide or combinations thereof. More preferred whitening pigment are particles comprising titanium dioxide, zinc oxide, zirconium oxide, mica, iron oxide or a combination thereof and most preferred pigment is titanium dioxide. The average diameter of the pigment is typical from 15 nm to 1 micron, more preferably from 35 nm to 800 nm, even more preferably from 50 nm to 500 nm and still even more preferably from 100 to 300 nm.

Particularly preferred moisturizing agents includes, petrolatum, aquaporin manipulating actives, oat kernel flour, substituted urea like hydroxyethyl urea, hyaluronic acid and/or its precursor N-acetyl glucosamine, hyaluronic acid and/or its precursor N-acetyl glucosamine, or a mixture thereof.

A wide variety of organic sunscreen is suitable for use in combination with the essential ingredients of this invention. Suitable UV-A/UV-B sunscreen include, 2-hydroxy-4-methoxybenzophenone, octyldimethyl p-aminobenzoic acid, digalloyltrioleate, 2,2-dihydroxy-4-methoxybenzophenone, ethyl-4-(bis(hydroxypropyl)) aminobenzoate, 2-ethylhexyl-2-cyano-3,3-diphenylacrylate, 2-ethylhexylsalicylate, glyceryl p-aminobenzoate, 3,3,5-trimethylcyclohexylsalicylate, methylanthranilate, p-dimethyl-aminobenzoic acid or aminobenzoate, 2-ethylhexyl-p-dimethyl-amino-benzoate, 2-phenylbenzimidazole-5-sulfonic acid, 2-(p-dimethylaminophenyl)-5-sulfonicbenzoxazoic acid, 2-ethylhexyl-p-methoxycinnamate, butylmethoxydibenzoylmethane, 2-hydroxy-4-methoxybenzophenone, octyldimethyl-p-aminobenzoic acid and mixtures thereof. The most suitable organic sunscreens are 2-ethylhexyl-p-methoxycinnamate, butylmethoxydibenzoylmethane or a mixture thereof.

Vitamin B3 compounds (including derivatives of vitamin B3) e.g. niacin, nicotinic acid or niacinamide are the preferred skin glowing agent as per the invention, most preferred being niacinamide.

Some compositions may include thickeners. These may be selected from cellulosics, natural gums and acrylic polymers but not limited by this thickening agent types. Amounts of thickener may range from 0.01 to 3% by weight of the active polymer (outside of solvent or water) in the compositions. Preservatives can desirably be incorporated into the compositions of this invention to protect against the growth of potentially harmful microorganisms.

Particularly preferred preservatives are phenoxyethanol, methyl paraben, propyl paraben, imidazolidinyl urea, sodium dehydroacetate and benzyl alcohol. The preservatives should be selected having regard for the use of the composition and possible incompatabilities between the preservatives and other ingredients. Preservatives are preferably employed in amounts ranging from 0.01% to 2% by weight of the composition.

A variety of other optional materials may be formulated into the compositions. These may include: antimicrobials such as 2-hydroxy-4,2′,4′-trichlorodiphenylether (triclosan), 2,6-dimethyl-4-hydroxychlorobenzene, and 3,4,4′-trichlorocarbanilide; scrub and exfoliating particles such as polyethylene and silica or alumina; cooling agents such as menthol; skin calming agents such as aloe vera; and colorants.

The composition may comprise water in amount of 10 to 95% by weight of the composition, more preferably from 25 to 90%, even more preferably from 32 to 85%, most preferably from 45 to 78% by weight of the composition.

Preferably, the composition has a viscosity of at least 10 mPa·s, more preferably in the range 30 to 10000 mPa·s, even more preferably 50 to 5000 mPa·s, and most preferably 100 to 2000 mPa·s, when measured at 20 degrees C. at a relatively high shear rate of about 20 s⁻¹. Preferably, the composition is in the form of fluid.

“Component” of the cosmetic refers to any ingredient in the cosmetic composition except water.

Preferably, the step of treating the skin surface with a cosmetic composition or the component thereof comprises topically applying the cosmetic composition onto the skin surface, preferably by human hand. The amount of the cosmetic composition is preferably 0.1 to 100 g, preferably 0.5 to 10 g for each time.

Preferably, the skin surface was treated by the cosmetic composition with a frequency of at least once a day, more preferably twice to four times a day. Preferably, such treatment continues for a duration of one week to one year, more preferably two weeks to three months.

The step of identifying a second individual microbiome network is conducted as same way as identifying the first individual microbiome network.

Preferably, the step of comparing the first and second individual microbiome networks may be conducted by comparing at least one of attributes selected from node, edge, node degree, clustering coefficient, and visualized microbiome network. Preferably, the visualized first and second individual microbiome networks are compared to indicate the difference. Alternatively or additionally, the node degrees of the first and second individual microbiome networks are preferably compared.

Preferably, the method comprises a further step of comparing the first, and/or second individual microbiome networks with a benchmark individual microbiome network. The benchmark individual microbiome network refers individual microbiome network of healthy skin conditions. The benchmark individual microbiome network may be obtained in any way, for example selecting a group of benchmark persons and obtaining an average benchmark individual microbiome network from skin surface of the group of benchmark persons. The benchmark person refers to a group of persons with healthy skin conditions in a sufficient number (greater than 10). Alternatively or additionally, the step of identifying a benchmark individual microbiome network comprising the step of finding a benchmark individual microbiome network from a database. Preferably, this database is formed through testing on a wide range of skin surfaces of healthy individuals, so as to produce a library in which the benchmark individual microbiome networks are contained.

The method of the present invention is particular effective when used to evaluate the efficiency of the cosmetic composition or the component relative to a placebo product. Thus, in a preferred embodiment the method comprises of treating the skin surface having the identified first individual microbiome network with placebo product in step (ii); and (iii) identifying a forth individual microbiome network on the skin surface that was treated by placebo product in step (iii); and (iv) comparing the second and forth individual microbiome networks. Alternatively, the personal care composition and placebo product may treat skin surfaces having different individual microbiome networks. Therefore, the method preferably comprises the steps of identifying a first and third individual microbiome networks of skin surfaces; treating the skin surfaces having the identified first and third individual microbiome networks with a cosmetic composition or the component thereof, and a placebo product respectively; identifying second and forth individual microbiome networks on the treated skin surfaces; and comparing the difference of first and second individual microbiome networks, and difference of third and forth individual microbiome networks.

Placebo as used herein means product which have no or lower levels of component than that of the personal care product or component to be tested. The placebo may be any composition different from the personal care product or component to be tested. However, it is preferred that the concentration of the component by weight of the placebo product is no greater than half (½), more preferably one quarter (¼), and most preferably one tenth ( 1/10) of the concentration of the component by weight of the personal care product or component to be tested. Most preferably the placebo is water (or at least comprises at least 99% water by weight of the placebo product, more preferably 99.9 to 100%).

The present invention also provides a method for preparing a recordable medium for demonstrating the evaluating result of a cosmetic composition or a component thereof comprises a step of capturing one or more images for the comparison and storing the same on a recordable medium. Recording medium comprises storage means having magnetic materials. Online or cloud storage is also included.

A method for demonstrating the evaluating result of a cosmetic composition or a component thereof comprising a step of displaying images of microbiome network Displaying images also include play a video. The image may be displayed in electric medium, paper, internet, programs and/or applications.

The following examples are provided to facilitate an understanding of the invention. The examples are not intended to limit the scope of the claims.

### EXAMPLES

### Example 1

This example showed that individual microbiome network is more sensitive and efficient than population microbiome network in demonstrating the efficiency of the skin care composition.

A group of 27 volunteers were recruited and given a commercial facial cleanser followed by a commercial facial cream to use twice a day for four weeks. Microbial samples were taken at baseline (0 week) and four weeks after product usage.

Facial microbiome samples were collected from upper cheeks using a cup scrub technique with phosphate buffered saline buffer (pH 7.9) containing 0.1% TritonX-100 (93443, Sigma, Missouri, USA). Buffer samples were stored at −80° C. before analysis. As controls, mock community, blank buffer control and PCR negative control samples were included. Microbial DNA was extracted from the samples using a DNA extraction kit (DNeasy Blood & Tissue kit, 69506, Qiagen, Hilden, Germany) following the manufacturer's instructions.

The microbial DNAs were sequenced at the variable region (V1-V2) of the 16S rDNA gene for bacterial classification. The V1-V2 region was amplified using a primer set (forward primer (SEQ ID NO 1): 5′-CCGAGTTTGATCMTGGCTCAG-3′ and reverse primer (SEQ ID NO 2): 5′-GCTGCCTCCCGTAGGAGT-3′), and sequenced by Beijing Genomics Institute (BGI, Wuhan, China) by using fusion primers with dual indices and adapters. The quantity and quality of the libraries were analyzed by Bioanalyzer (Agilent Technologies, California, USA). Only qualified libraries were used for sequencing on the Illumina Miseq PE300 platform, initially resulting in approximately 159 million raw sequence paired reads. Sequences with low quality were discarded before analysis.

Microbiome composition was generated from clean sequencing raw data by QIIME (Quantitate Insights into Microbial Ecology) version 1.9.1. Taxonomy classification was carried out using a Lowest Common Ancestor methodology against the following databases: SILVA, NCBI, RDP, DDBJ, Greengenes, CAMERA, EMBL, EzTaxon. 143 million overlapping contigs were grouped into 729 OTUs. Microbiome composition of each sample were accumulated by sequencing counts according to taxonomy classification. Before microbiome network construction, OTUs that had frequencies of less than 80% in samples in each group were removed.

The population microbiome network for the group of volunteers was calculated and obtained by performing SPIEC-EASI, using the neighborhood selection method with a minimum λ threshold of 0.01. All steps were computed using the R package SPIEC-EASI (version 1.0.7).

The individual microbiome network for each sample was obtained by follow procedures:

Step-1: input the sequencing data (relative abundances of species: x, y, z, . . . ) with N samples of p species. Let q=1. N is the number for all samples and q is the sequence number of a specific sample.

Step-2: calculate each partial correlation r_{xy,z}^{(N)} with N samples using SPIEC-EASI.

Step-3: calculate each partial correlation r_{xy,z}^{(N/q)} with N−1 samples by removing the qth sample using SPIEC-EASI.

Step-4: calculate qth sample's specific partial correlation for each pair of species x and y by following the equation:

$${I}_{xy , z}^{( q )} = {Nr}_{xy , z}^{( N )} - ( N - 1 ) {r}_{xy , z}^{( N / q )}$$

Step-5: Let q=q+1, and go to Step-3 until q=N. Partial correlations of all pairs for each sample form the individual microbiome network of an individual.

The effects of commercial skin care products were evaluated by different kinds of microbiome networks. The two population microbiome networks of the group of samples were constructed before and after product intervention. The individual microbiome network for each sample was also constructed before and after product intervention.

To quantitively demonstrate the efficiency of population and individual microbiome networks, node degrees (attribute to describe the connectivity of a network) for microbiome networks were calculated.

TABLE 1

| | Node degree of MNP | Averaged node degree of MNIs |
|---|---|---|
| 0 Week | 0.42 | 1.91 |
| 4 Weeks | 0.43 | 2.13^{a} |

Significant difference from average node degree of MNIs at 0 week(p < 0.05)

Table 1 shows the node degrees for microbiome networks. The middle column indicates the node degrees of group microbiome network at 0 week (before product intervention) and 4 weeks (after product intervention). They are 0.42 and 0.43 respectively, indicating the product efficacy is not able to be demonstrated by the node degrees of MNP. In contrast, the right column shows the average node degrees of MNIs of all each sample at 0 week and 4 weeks. There is statistically significant difference between node degrees before and after product intervention.

It is evident from Table 1 that individual microbiome network is more sensitive and efficient than population microbiome network in demonstrating the efficiency of personal care products.

### Example 2

This example demonstrates the sensitivity of the method of assessing cosmetic composition of the present invention.

### (a) Classification by Image Analysis

The ASIA CR® images (Canfield Scientific, Inc. USA) were used to assess the facial skin of the individuals. A skin age of the specific object was obtained by the image and the measuring system. When the measured skin age is higher than the actual age of the subject, they are called “bad ager”. When the measured skin age is lower than the actual age of the subject, they are called “good ager”.

### (b) Classification by Microbiome Analysis

Two methods, species relative abundance, and MNI according to the present invention were compared to classify the subjects in the test group. A “good ager” benchmark group (35 subjects) and 3 independent test groups (26 subjects each) with different product interventions were selected for discrimination analysis. Subjects in each group were recruited and given a commercial facial cleanser followed by a commercial facial cream to use twice a day for four weeks. Microbial samples were taken at baseline (0 week) and four weeks after product usage for test groups. The species relative abundance for each individual subject and MNIs for benchmark group and test groups were obtained by following same procedure as described in Example 1. The discrimination analysis (by software SAS JMP 14) was employed to compare the classification sensitivity of species relative abundance and MNI. The classification for each subject were conducted by comparing the species relative abundance (or MNI) in test group and benchmark group and be identified “bad ager” or “good ager”. If the classification is contradicted to the classification by image analysis, it is considered as misclassification.

The results for discrimination analysis were shown in Table 2.

TABLE 2

Misclassified percentage (%)

| Product Intervention | Species relative abundance | MNI |
|---|---|---|
| 1 | 5.8 | 0 |
| 2 | 3.7 | 0 |
| 3 | 3.8 | 0 |

As indicated in Table 2, MNI is more sensitive and accurate method to reflect the real condition of the skin than the species relative abundance and therefore a more sensitive and accurate method for assess cosmetic composition.

1. A method for evaluating a cosmetic composition or a component thereof, comprising the steps of:
(i) identifying a first individual microbiome network on a skin surface of a person;
(ii) treating the skin surface having the identified first individual microbiome network with a cosmetic composition or the component thereof;
(iii) identifying a second individual microbiome network on the skin surface that was treated; and
(iv) comparing the first and second individual microbiome networks,
wherein identifying a first individual microbiome network comprises the steps of:
(a) obtaining a first population microbiome network from a group of samples consisting of N individuals including said person, wherein N is an integer of at least 3; (b) obtaining a second population microbiome network from the same group of samples without the sample from said person; and
(c) determining the first individual microbiome network of said person by subtracting effects of the second population microbiome network from the first population microbiome network.

2. The method according to claim 1 wherein obtaining a population microbiome network comprises the step of determining microbiome composition data of the desired skin surface; and generating a population microbiome network by representing microbial organisms in each matrix as a network of plurality of nodes corresponding the group of samples.

3. The method according to claim 2 wherein the microbiome composition comprises a set of taxa comprising at least one of: Marvinbryantia (genus), Erysipelotichales (order), Erysipelotrichia (class), Bacteroidetes (phylum), Staphylococcus (genus), Staphylococcaceae (family), Bacillales (order), Actinobacteria (class), Firmicutes (phylum), Actinobacteria (phylum), and Propionibacterium (genus).

4. The method according to claim 1 wherein the skin surface has at least one undesired skin condition selected from dry skin, aging, wrinkle, darkness, acne, spots, dandruff, and weak skin barrier.

5. The method according to claim 1 wherein the step of comparing the first and second individual microbiome networks is conducted by comparing at least one attribute selected from node, edge, node degree, clustering coefficient, and visualized microbiome network.

6. The method according to claim 1 wherein the method further comprises the steps of identifying first and third individual microbiome networks of skin surfaces; treating the skin surfaces having the identified first and third individual microbiome networks with the cosmetic composition or the component thereof, and a placebo product respectively; identifying second and forth individual microbiome networks on the treated skin surfaces; and comparing a difference of the first and second individual microbiome networks, and a difference of the third and forth individual microbiome networks.

7. A method for preparing a recordable medium for demonstrating an evaluating result of a cosmetic composition or a component thereof, the method comprising the steps of:
(i) identifying a first individual microbiome network on a skin surface of a person;
(ii) treating the skin surface having the identified first individual microbiome network with a cosmetic composition or the component thereof;
(iii) identifying a second individual microbiome network on the skin surface that was treated; and
(iv) comparing the first and second individual microbiome networks, wherein the method further comprises a step of capturing one or more images for the comparison and storing the image or images on a recordable medium,
further wherein identifying a first individual microbiome network comprises the steps of:
(a) obtaining a first population microbiome network from a group of samples consisting of N individuals including said person, wherein N is an integer of at least 3; (b) obtaining a second populational microbiome network from the group of samples without the sample from said person; and
(c) determining the first individual microbiome network of said person by subtracting effects of the second population microbiome network from the first population microbiome network.

8. A method for demonstrating the evaluating result of a cosmetic composition or a component thereof comprising a step of displaying images of a microbiome network, wherein the images are captured and stored for:
(i) identifying a first individual microbiome network on a skin surface of a person;
(ii) treating the skin surface having the identified first individual microbiome network with a cosmetic composition or the component thereof;
(iii) identifying a second individual microbiome network on the skin surface that was treated; and
(iv) comparing the first and second individual microbiome networks,
wherein identifying a first individual microbiome network comprises the steps of:
(a) obtaining a first population microbiome network from a group of samples consisting of N individuals including said person, wherein N is an integer of at least 3; (b) obtaining a second population microbiome network from the group of samples without the sample from said person; and
(c) determining the first individual microbiome network of said person by subtracting effects of the second population microbiome network from the first population microbiome network.


SEQUENCE LISTING (2 sequences)

SEQ ID NO: 1 - DNA, 21 bp, unidentified
  source 1..21: mol_type = unassigned DNA
ccgagtttgatcmtggctcag

SEQ ID NO: 2 - DNA, 18 bp, unidentified
  source 1..18: mol_type = unassigned DNA
gctgcctcccgtaggagt